(19) United States
(12) Patent Application Publication
(10) Pub. No.: US 2009/0130144 A1
(43) Pub. Date: May 21, 2009

(54) Direct vaccination of the bone marrow

(75) Inventors: Scott E. Strome, Reisterstown, MD (US); Xiaoyu Zhang, Catonsville, MD (US)

Correspondence Address: BROWDY AND NEIMARK, P.L.L.C.;624 NINTH STREET, NW, SUITE 300, WASHINGTON, DC, 20001-5303, US

(73) Assignees: University of Maryland, Baltimore, Baltimore, MD (US); Mayo Foundation For Medical Research, Rochester, MN (US)

(21) Appl. No.: 11/911,618

(22) PCT Filed: Apr. 14, 2006
(86) PCT No.: PCT/US06/14409

§ 371 Date: Jun. 10, 2008

Related U.S. Application Data
(60) Provisional application No. 60/671,473, filed on Apr. 15, 2005.

(51) Int. Cl. A61K 39/12; A61K 39/00; A61K 39/07; C12N 5/08

(52) U.S. Cl. 424/206.1; 424/184.1; 424/204.1; 424/246.1; 424/218.1; 435/372.3

(57) ABSTRACT

The present invention provides methods for eliciting an effective immune response against a weakly immunogenic disease or for priming T cells to become memory T cells against a weakly immunogenic disease by directly vaccinating into the bone marrow of the patient an antigen associated with the weakly immunogenic disease. Also included in the present invention is an isolated population of human memory CD8⁺ T cells from the bone marrow which is in a heightened activation state with a unique effector phenotype.

### CROSS-REFERENCE TO RELATED APPLICATIONS

The present application claims the benefit of priority under 35 U.S.C. §119(e) from provisional U.S. application No. 60/671,473, filed Apr. 15, 2005, the entire content of which is herein incorporated by reference.

### BACKGROUND OF THE INVENTION

1. Field of the Invention

The invention relates to immunization against weakly immunogenic diseases such as malignancies and some infectious agents.

2. Description of the Related Art

Memory T cells are defined by their capacity to mount a rapid response to secondary antigenic challenge (Veiga-Fernandes et al., 2000), and their ability to maintain homeostatic proliferation in the absence of antigenic stimulation (Kaech et al., 2001 and 2002). Recently, memory T cells have been categorized into effector memory (T_{EM}), CD45RO⁺CD62L^{Low}CCR7^{Low}, and central memory (T_{CM}), CD45RO⁺CD62L^{Hi}CCR7^{Hi}, subsets based on both their homing characteristics and effector functions (Sallusto et al., 1999). While T_{CM} are primarily distributed in lymphoid tissue, T_{EM} can traffic to and reside in diverse non-lymphoid sites, including lung, liver and intestine (Masopust et al., 2001). Whether residence in a particular anatomic compartment confers distinct phenotypic or functional properties on the indigenous memory T cells has not been shown.

The bone marrow (BM) represents the primary site of hematopoiesis and a rich source of stem cell progenitors. Human BM also contains mature lymphocytes, including T lymphocytes, B lymphocytes, and antibody-producing plasma cells. However, the role of BM-derived T lymphocytes in the peripheral immune response is poorly understood. Recent evidence suggests that residence in a particular anatomic compartment, e.g., BM might confer distinct phenotypic or functional properties on the indigenous memory T cells. Early studies in tumor models found that the presence of live tumor cells in the BM was associated with systemic protection from tumor specific challenge. In addition, tumor cells in BM were controlled in a dormant state by CD8⁺ T cells (Khazaie et al., 1994; and Muller et al., 1998). Correlate data from patients with breast cancer demonstrated that, following adoptive transfer, primed T cells from the BM, but not the peripheral blood (PB), can effectively treat autologous breast cancer xenografts in NOD/SCID mice (Feuerer et al., 2001; Beckhove et al., 2004; and Bai et al., 2003). Similar findings have recently been described for pancreatic tumors (Schmitz-Winnenthal et al., 2005), myeloma (Choi et al., 2005, and melanoma (Letsch et al., 2003). Furthermore, in mouse viral infection models, the BM was found to harbor virus-specific memory CD8⁺ T cells that could mediate protection from lymphocytic choriomenigitis virus (LCMV) infection when adoptively transferred into naïve SCID hosts (Slifka et al., 1997), and virus-specific memory CD8⁺ T cells were also produced in BM in response to VSV infection (Masopust et al., 2001).

Feuerer and coworkers reported that naive, antigen-specific T cells home to bone marrow, where they can be primed (Feuerer et al., 2003). It is suggested that bone marrow contains a microenvironment that allows interactions between antigen-presenting denditic cells and naive, circulating antigen-specific T cells, leading to the induction of primary and memory CD8⁺ T-cell response.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention provides a method for eliciting an effective immune response against a disease which normally generates either no immune response or a weak and insufficient immune response in a patient suffering from the disease. This method involves administering directly into the bone marrow of the patient an antigen associated with the disease or associated with the causative agent of the disease to elicit an effective immune response against the weakly immunogenic disease.

The present invention also provides a method for priming T cells in the bone marrow to become memory T cells against a disease which normally generates a weak and insufficient immune response in a patient exposed to the causative agent of the disease. This priming method involves administering directly into the bone marrow of the patient but with an antigen associated with a disease or associated with a causative agent of a disease which the patient has not previously been exposed. Accordingly, this method primes T cells against the weakly immunogenic disease so that upon a subsequent encounter with an antigen associated with the disease or the causative agent of the disease, the primed memory T cells can be quickly activated to raise an effective immune response.

The present invention further provides an isolated population of human memory T cells from the bone marrow which is in a hyper-responsive/heightened activation state and which demonstrate a unique effector phenotype.

### BRIEF DESCRIPTION OF THE DRAWINGS

FIGS. 1A-1J show representative FACS analyses from one patient. The expression of the costimulatory and activation associated molecules CD45RA (FIG. 1A), CD45RO (FIG. 1B), CD62L (FIG. 1C), CD27 (FIG. 1D), CD28 (FIG. 1E), CD25 (FIG. 1F), CD38 (FIG. 1G), CD69 (FIG. 1H), HLA-DR (FIG. 1I), and CD57 (FIG. 1J), were compared between CD8⁺ T cells from PB (shaded area) and BM (open thick line). Open solid line indicates the isotype control.

FIGS. 2A-2E show that virus specific CD8⁺ T cells in the bone marrow (BM) have increased potential for degranulation and cytokine release in response to recall antigens versus those in PB. Mononuclear cells from the PB (▪) and BM (□) were stimulated in vitro for 5 hrs with CEF peptide cocktails (FIGS. 2A and 2B) or 25 ng/ml PMA plus 1 μg/ml ionomycin (FIGS. 2C and 2D) in the presence of CD107a mAb and monensin. Samples were then stained for intracellular IFN-γ, CD3, and CD8, and analyzed by flow cytometry. The results are shown as the percentage of CD107a (FIGS. 2A and 2C) or IFN-γ (FIGS. 2B and 2D) positive cells within CD3⁺CD8⁺ gates. FIG. 2E shows a representative FACS analysis from one patient sample. The background frequencies of CD107a expression in BM and PB were 0.13±0.05% (range 0.06-0.22%) and 0.09±0.05% (range 0.03-0.14%), respectively. The background frequencies of IFN-γ production in CD8⁺ T cells from BM and PB were 0.03±0.02% (range 0.00-0.08%), and 0.01±0.02% (range 0.00-0.04%), respectively (data not shown). The results shown are presented after subtracting for background.

FIGS. 3A and 3B show the identification of CMV-specific CD8⁺ T cells by pentamer staining from PBMCs and BMMCs. In FIG. 3A, lymphocytes from BM and PB were stained with HLA-A2/CMV pentamer and cell surface molecules. Results are from five patients with detectable pentamer staining and one patient with undetectable staining, as negative control. Cells were gated on CD3⁺CD8⁺ lymphocytes. Gated populations are plotted as CD8 staining versus pentamer staining. The numbers represent the percentage of pentamer positive cells within the CD8⁺ T lymphocyte population. Representative patient samples are presented in FIG. 3B.

FIGS. 4A and 4B are graphs showing comparison of perforin, granzyme B and Fas L expression in BM and PB CD8⁺ T cells as percentage of positive cells. The expression of perforin, granzyme B and Fas L were compared between PB and BM CD8⁺ T cells (FIG. 4A). Data represent the mean (±SD) from 7 patients. *p<0.05. FIG. 4B shows a comparison of perforin, granzyme B and Fas L expression on anti-CD3 activated PB and BM CD8⁺ T cells. PBMCs and BMCs were stimulated with 0.5 μg/ml of coated anti-CD3 mAb for 3 days. Cells were then stained for intracellular perforin, granzyme and surface Fas L and other surface markers, and analyzed by flow cytometry. A representative experiment is shown (n=3).

FIGS. 5A and 5B show that the BM contains an increased population of CD8⁺ T_{EM} cells versus PB. PBMCs and BMMCs were isolated from 7 OA patients and CD8⁺ T cells were analyzed for the expression of CD45RO and CD62L by four color flow cytometry. In FIG. 5A, a representative sample is shown as a plot of CD45RO expression versus CD62L expression. FIG. 5B shows the percentage of T_{CM} (□) and T_{EM} (▪) CD8⁺ lymphocytes from BM and PB. FIGS. 5C and 5D are graphs showing that the phenotype of BM CD8⁺ T_{EM} cells differs from those in the periphery. Cell surface expression of costimulatory and activation and function associated molecules on T_{EM} (FIG. 5C) and T_{CM} (FIG. 5D) of PB (▪) and BM (□) were analyzed by 4 color flow cytometry. T_{EM} and T_{CM} subsets were gated according to the expression of CD62L on CD8⁺CD45RO⁺ cells. Results are shown as mean±s.e.m. (n=7). *P<0.05.

FIGS. 6A-6C show that the T_{EM} component contributes to the augmented antigen specific CD8 response to recall antigen in the BM. In FIG. 6B, PBMCs (▪) and BMMCs (□) from 5 donors were incubated with 2 μg/ml of CEF peptides for 5 hours, then analyzed for CD107a mobilization. In FIG. 6A, a representative sample is shown as FACS plot. Lymphocytes were identified by forward scatter and side scatter. Lymphocytes were further gated on CD8⁺CD45RO⁺, and CD107a expression was plotted against CD62L expression. The numbers in the upper left and upper right corner represent the frequencies of CD107a positive cells within CD62L⁻ and CD62L⁺ memory CD8⁺ T cells. In FIG. 6C, direct cytotoxic activity of T_{EM} and T_{CM} of PB and BM was compared in T cell lines generated from 3 patients. Representative data from one patient is shown. Statistical analysis of the pooled data confirmed that cytotoxicity was significantly higher in the BM T_{EM} subset than in PB T_{EM} subset.

FIGS. 7A-7E are graphs showing the expression of the receptors for chemokines and IL-7 and IL-15 by BM CD8⁺ T_{EM} cells. The surface expression of chemokine receptors, CXCR4 (FIG. 7A), CCR5 (FIG. 7B) and CXCR1 (FIG. 7C), and α chain of IL-7R (FIG. 7D) and IL-15R (FIG. 7E) were determined by 4-color flow cytometric analysis of 6 patients. Results are shown as mean±SD. *P<0.05

### DETAILED DESCRIPTION OF THE INVENTION

Human bone marrow (BM) contains mature T and B lymphocytes, yet its role in the peripheral immune response to human viral infections remains poorly defined. The present inventors have discovered that human BM contains a novel functionally enhanced population of memory CD8⁺ T cells which can serve as a platform for immunotherapy, using either direct compartmental boosting or adoptive transfer approaches. The present inventors sought to investigate the phenotypic signature and effector function of memory T cells in human BM, isolated from a cohort of patients with degenerative joint disease. The results obtained by the present inventors and presented in the Example hereinbelow define a distinct population of CD8⁺ effector memory (EM) T cells which exist in the BM of patients with osteoarthritis (OA), and demonstrate a rapid and profound response to challenge with recall antigens. These cells maintain a unique phenotype, a specific antigenic signature, expressing high levels of CD27, CD28, CD38, CD69, and HLA-DR and low levels of CD57. These cells exhibit a profound recall response to viral antigens and display unique patterns of perforin and granzyme B regulation in response to TCR stimulation. The enhanced recall response indicates that the BM serves as a repository for these “super memory” effector T cells which are in an intrinsically heightened/hyper-responsive activation state. The functionally enhanced population of memory CD8⁺ T cells also exhibit enhanced cytolytic activity.

The present invention therefore makes use of the present inventors' discovery of this population of “super memory” effector T cells in the BM by providing a method for eliciting an effective immune response against a disease, which normally generates a weak and insufficient immune response in a patient suffering from the disease, by direct administration of a suitable antigen into the bone marrow of the patient (i.e., thin bore needle inserted into the hip). This method takes advantage of the presence of the population of effector memory T cells in the BM that have previously encountered an antigen, such as an antigen associated with a disease which normally generates a weak and insufficient immune response or associated with the causative agent of the disease, and are in an intrinsically heightened state capable of rapidly responding to challenge by recall antigens.

Memory T cells are T cells which have been exposed to antigen and survive for extended periods in the body in a resting state without the presence of stimulating antigen. These memory T cells are more responsive to “recall” antigens when compared with the naive T cell response to antigen. The population of T cells discovered by the present inventors are however designated “super memory” because they are in an intrinsically heightened or hyper-responsive activation state capable of instantly responding and eliciting an effective immune response against the recall antigen.

The antigen in the present method for eliciting an effective immune response is a recall antigen associated with a weakly immunogenic disease or a causative agent of the weakly immunogenic disease. By being a recall antigen, this means that the “super memory” T cells in the patients' bone marrow have previously encountered the antigen and are ready to mount an immune response upon subsequent exposure to the antigen; otherwise, the present method would instead be priming T cells.

It is intended that the term “weakly immunogenic disease” be those diseases which normally generate a weak and insufficient immune response in a patient suffering from the disease. Non-limiting examples of such weakly immunogenic diseases are various types of cancers, viral infections, and diseases caused by agents of bioterrorism. The various types of cancer include, but are not limited to, breast cancer, colon cancer, prostate cancer, lung cancer, brain cancer, head and neck cancer, melanoma, sarcomas, etc. In the case of cancer, the antigen is a tumor associated antigen (TAA) or a peptide fragment thereof. Viral infections include but are not limited to infections with cytomegalovirus, Epstein Barr virus, HIV, bird (avian) flu viruses (e.g., strain H5N1), influenza virus, etc. A preferred embodiment of a viral infection as a weakly immunogenic disease for purposes of the present invention is influenza, particularly in the elderly, a susceptible and vulnerable population which cannot respond as effectively to flu vaccines. Agents of bioterrorism are the causative agents of diseases such as anthrax and hemorrhagic fevers (ebola and other hemorrhagic viruses), etc., which may have been encountered by the patient just recently.

In the event that a patient has not had prior exposure to the antigen, the present invention provides a method for priming T cells to become memory T cells in the bone marrow against a weakly immunogenic disease. Similar to the method for eliciting an effective immune response, an antigen associated with the causative agent of the weakly immunogenic disease is administered directly into the bone marrow of a patient to prime T cells in the bone marrow. The causative agent and the weakly immunogenic disease are among those disclosed above for the method for eliciting an effective immune response, such as influenza, but the causative agent is preferably an agent of bioterrorism to which the patient could later be potentially exposed, such as but not limited to Bacillus anthracis or its biotoxin, and the ebola virus or another hemorrhagic virus.

The antigen(s) administered to a patient include purified or partially-purified preparations of protein, peptide, carbohydrate or lipid antigens. Any tumor associated antigen can be considered as an antigen for the purposes of the present invention.

The antigen can be administered as part of a vaccine preparation. While the term “vaccine” is often used to refer only to vaccinations intended to induce prophylaxis, this term is intended to include vaccination for therapeutic purposes as well. For example, vaccines that include tumor-associated antigens are intended to elicit an immune response against tumors/cancers. Vaccines to viral particles may be used not only to create prophylaxis against the virus, but also to eradicate an existing viral infection. For example, vaccines are available against HBV and others against AIDS and HCV, which are in active development. Thus, the term “vaccine” applies to the administration of any antigen for the purpose of eliciting an immune response against that antigen or to a cross-reactive antigen that exists in situ. Suitable vaccines include an influenza, smallpox, anthrax, hepatitis B virus, human papilloma virus, herpes simplex virus, polio, tuberculosis or anti-cancer vaccine. Non-limiting examples of viral antigen recall peptides that can be used include those peptides listed in Table 1 for cytomegalovirus, Epstein Barr virus and influenza virus.

The amount of antigen(s) present in each vaccine dose, is selected as an amount capable of eliciting a protective immune response in vaccinated subjects. This amount will depend on the specific antigen and the possible presence of typical adjuvants, and can be identified by a person skilled in the art. In general, each dose will contain 1-1000 micrograms of antigen, preferentially 10-200 μg. Further components can also be advantageously present in the vaccine.

The vaccine composition can be formulated in any conventional manner, as a pharmaceutical composition comprising sterile physiologically compatible carriers such as saline solution, excipients, adjuvants, preservatives, stabilizers, etc.

The vaccine can be in a liquid or in lyophilized form, for dissolution in a sterile carrier prior to use.

The pharmaceutically acceptable carrier, excipient, diluent or auxiliary agent can be easily identified accordingly for each formulation by a person skilled in the art.

The method of the present invention can be used in both prophylactic and therapeutic treatment of infectious diseases and cancer. In particular, the method can be used for preventing (i.e., prophylactic vaccines) as well as for the treatment of (i.e., therapeutic vaccines) viral diseases. Moreover, the method can also be used in the prevention/inhibition and treatment of cancer or other diseases when suitable antigens are used. This can be achieved by using antigens against infectious agents associated with human malignancies, e.g., EBV, HPV and H. pilori, or well defined tumor associated antigens such as those characterized in human melanoma, e.g., MAGE antigens, thyrosinase gap100, and MART, as well as in other human tumors. For example, WO 00/06723 discloses tumor associated antigens and peptide antigens thereof for numerous types of cancers, i.e., mucin (i.e., MUC-1), Lactadherin and Her2/neu for breast carcinomas, prostate specific antigen (PSA), prostate specific membrane antigen (PSMA), and prostate acid phosphatase (PAP) for carcinoma of the prostate (CAP), uroplakins for transitional cell carcinoma (TCC), and CRIPTO-1 (teratocarcinoma-derived growth factor).

For squamous cell carcinoma of the head and neck (SCCHN), the MAGE-A3 differentiation antigen (SEQ ID NO:33; von der Bruggen et al., 1991), initially identified in the human MZ2E melanoma, and the human papilloma virus (HPV) 16 E7 nuclear protein (SEQ ID NO:39), recently identified by the laboratory of the present inventors, as well as others, as an independent risk factor for oropharyngeal SCC (Strome et al., 2002; Gillison et al., 1999 and 2000), are further examples of tumor associated antigens for the purposes of the present invention. Peptide epitopes of MAGE-A3 for use in immunotherapy for cancer include SEQ ID NOs:34-38. Similarly, peptide epitopes of HPV 16 E7 nuclear protein for use in immunotherapy for cancer include SEQ ID NOs:40-42. The laboratory of the present inventors have also developed large synthetic peptides, up to 50 amino acid residues in length, which contain multiple epitopes linked to a translocating region (SEQ ID NO:43) of HIV TAT. These “Trojan antigens” avoid any possible issue of proteolysis because they can be internalized and processed. The laboratory of the present inventors has also established that multiple T cell epitopes can be joined together using furin-cleavable linkers (SEQ ID NO:44), which allow the release of the individual epitopes in the Golgi, where the furin endopeptidase residues. Preferred examples of such Trojan antigens are the MAGE-A3 Trojan antigen of SEQ ID NO:45 and the HPV16E7 Trojan antigen of SEQ ID NO:46.

Having now generally described the invention, the same will be more readily understood through reference to the following example which is provided by way of illustration and is not intended to be limiting of the present invention.

### EXAMPLE

The identification and characterization of a novel functionally enhanced “super memory” CD8⁺ T cell population in human BM isolated from patients undergoing total joint replacement for osteoarthritis (OA) is reported in this example. These BM-derived memory CD8⁺ T cells differ strikingly from memory CD8⁺ T cells in peripheral blood (PB), expressing elevated levels of CD27, HLA-DR, CD38, and CD69, unique patterns of chemokine receptors and expressing reduced levels of CD62L and CD57. Moreover, compared to the effector-memory subset (TEM) in PB, BM CD8⁺ T cells demonstrate a more vigorous recall response and express even higher levels of CD107a in response to pooled viral antigen (CEF) recall peptides. Interestingly, while BM T_{EM} have low levels of resting perforin and granzyme B, these molecules evidence profound upregulation in response to TCR stimulation. The results here reveal that human BM serves as a repository for unusually responsive memory CD8⁺ T cells that have therapeutic utility in tumor immunity and vaccine development.

### Materials and Methods

### Specimen Procurement

Entry criteria for this study included a diagnosis of osteoarthritis (OA) requiring a total joint arthroplasty, and the absence of known immunosuppression, autoimmune disease, and cancer (other than non-melanoma skin cancer). The protocol was approved by the Mayo Clinic College of Medicine Institutional Review Board and all patients signed written informed consent. Fifty ml of PB were collected and approximately 10 ml of BM was obtained at the time of surgery, while the patients were under anesthesia. The BM was collected directly from the medullary canal of the femur during bone preparation for total hip arthroplasty. The marrow was collected with a suction, syringe or receptacle as it was forced out of the canal with reaming or broaching instruments.

### Cell Preparation

Mononuclear cells from PB and BM were isolated by Ficoll-Paque (Amersham Biosciences, Uppsala, Sweden). The cells were used directly for flow cytometry or cultured with antigens and mitogens, or cryopreserved for future experimental analysis. For isolation of CD8⁺ T_{EM} and T_{CM} cells, mononuclear cells from PB and BM were stained with mouse anti-human monoclonal antibodies (mAbs) against CD8, CD45RO, and CD62L. CD8⁺ T_{EM} cells and T_{CM} cells were isolated using a FACSVantage SE with CellQuest Pro software (BD Biosciences). Purity was evaluated by post sort flow cytometry. Sorted cells exhibited a purity of at least 98%.

Isolated CD8⁺ T_{EM} and T_{CM} cells were expanded using a rapid expansion protocol described previously (Crossland et al., 1991). Briefly, T cells were cultured with 30 ng/ml anti-CD3 antibody (OrthoClone OKT3; Ortho Diagnostics, Raritan, N.J.) and 1000 u/ml IL-2 (Proleukin; Chiron, Emeryville, Calif.) in the presence of irradiated (30 Gy), allogeneic PBMC as feeder cells at a concentration of 1×10⁶/ml. After 14 days, cells were harvested and used for experiments or cryopreserved.

### Synthetic CEF Peptides

A panel of 32 8-11 mer cytomegalovirus, Epstein Barr virus and influenza virus (CEF) peptides (Table 1; Currier et al., 2002) were synthesized by Mayo Protein Core Facility. Purity was determined by HPLC and mass spectrophotometry. The peptides were dissolved in DMSO at 10 mg/ml and a peptide pool was made at a concentration of 100 μg/ml for each peptide.

TABLE 1

Synthetic CEF Peptides
Peptide sequences and HLA restriction

| | | Peptide | |
|---|---|---|---|
| HLA Allele | Virus | Sequence | SEQ ID NO: |
| A1 | Influenza A | VSDGGPNLY | SEQ ID NO:1 |
| | Influenza A | CTELKLSDY | SEQ ID NO:2 |
| A2 | Influenza M | GILGFVFTL | SEQ ID NO:3 |
| | Influenza A | FMYSDFHFI | SEQ ID NO:4 |
| | EBV LMP2A | CLGGLLTMV | SEQ ID NO:5 |
| | EBV BMLF1 | GLCTLVAML | SEQ ID NO:6 |
| A0201 | HCMV pp65 | NLVPMVATV | SEQ ID NO:7 |
| AA68 | Influenza NP | KTGGPIYKR | SEQ ID NO:8 |
| A3 | Influenza NP | RVLSFIKGTK | SEQ ID NO:9 |
| | Influenza A | ILRGSVAHK | SEQ ID NO:10 |
| | EBV | RVRAYTYSK | SEQ ID NO:11 |
| | EBV | RLRAEAQVK | SEQ ID NO:12 |
| A3, A11, | Influenza M | SIIPSGPLK | SEQ ID NO:13 |
| A60881 | | | |
| A11 | EBV EBNA4NP | AVFDRKSDAK | SEQ ID NO:14 |
| | EBV | IVTDFSVIK | SEQ ID NO:15 |
| | EBV | ATIGTAMYK | SEQ ID NO:16 |
| A24 | EBV RTA | DYCNVLNKEF | SEQ ID NO:17 |
| B7 | Influenza NP | LPFDKTTVM | SEQ ID NO:18 |
| | EBV | RPPIFIRRL | SEQ ID NO:19 |
| B8 | Influenza NP | ELRSRYWAI | SEQ ID NO:20 |
| | EBV B2LF-1 | RAKFKQLL | SEQ ID NO:21 |
| | EBV EBNA3A | FLRGRAYGL | SEQ ID NO:22 |
| | EBV EBNA3 | QAKWRLQTL | SEQ ID NO:23 |
| B18 | HCMV | SDEEEAIVAYTL | SEQ ID NO:24 |
| B27 | Influenza NP | SRYWAIRTR | SEQ ID NO:25 |
| | Influenza M | ASCMGLIY | SEQ ID NO:26 |
| | EBV EBNA3C | RRIYDLIEL | SEQ ID NO:27 |
| B35 | EBV EBNA3A | YPLHEQHGM | SEQ ID NO:28 |
| | CMVpp65 | IPSINVHHY | SEQ ID NO:29 |
| B44 | EBV | EENLLDFVRF | SEQ ID NO:30 |
| | HCMV | EFFWDANDIY | SEQ ID NO:31 |
| B0702 | HCMV | TPRVTGGGAM | SEQ ID NO:32 |

### HLA Typing

Genomic DNA was extracted from whole blood of patients using QIAamp DNA blood mini kit (Qiagen, Valencia, Calif.). HLA Class I typing was performed with Biotest HLA-A SSC kit (Biotest, Germany).

### Antibodies and Peptide MHC Class I Pentamer

Peridinin chlorophyll (PerCP) labeled anti-CD3, allophytocyamin (APC) labeled anti-CD8, phycoerythrin (PE) or fluorescein isothiocyanate (FITC) labeled anti-CD45RO, PE-Cy5 labeled anti-CD62L, PE labeled anti-CD25, CD38, CD69, HLA-DR, CCR5, CCR7, CXCR1, FITC labeled anti-CD45RA, CD57 and isotype control mAbs were purchased from BD Pharmingen (San Diego, Calif.). PE labeled anti-CD27, CD28 and CXCR4 were obtained from Ebioscience (San Diego, Calif.). PE labeled donkey anti-goat IgG were obtained from Jackson ImmuneResearch Laboratories (West Grove, Pa.). APC labeled HLA-A0201/NLVPMVATV pentamer was purchased from Proimmune (Springfield, Va.).

### Flow Cytometry

PBMCs and BMMCs were incubated with the mAbs for 30 min at 4° C., washed in PBS with 0.5% BSA (pH 7.0), and fixed in PBS with 2% paraformaldehyde. Cells were analyzed on a FACSCalibur with CellQuest software (Becton Dickinson, Mountain View, Calif.). For IL-15Rα, cells were first stained with specific antibody, washed and treated with PE labeled secondary antibody. Subsequently, IL-15Rα stained cells were stained with anti-CD8, anti-CD45RO and anti-CD62L mAbs. Labeled Cells were analyzed on a FACSCalibur with CellQuest software or LSRII with FACSDiva Software (Becton Dickinson, Mountain View, Calif.). Peptide HLA pentamer staining was performed according to manufacturer's protocol. 1×10⁶ cells were incubated with 10 μL of APC labeled pentamer for 45 mins at 4° C., washed in PBS twice, followed by the incubation with FITC labeled anti-CD8 for 30 mins. At least 5×10⁵ events were collected for each sample.

### CD107a and Intracellular IFN-γ Staining

CD107a staining was performed as recently described with a few modifications (Betts et al., 2003). Lymphocytes were stimulated in vitro with 2 μg/ml of the CEF peptides or mitogens in the presence of monesin A (Sigma) and FITC-conjugated mAbs for CD107a or isotype control (BD Pharmingen) for 5 hr. Cells were then harvested, washed, and stained for other surface molecules or permeabilized and stained for intracellular IFN-γ. Spontaneous CD107a expression and/or cytokine production, in the absence of peptide stimulation, was included as a negative control.

To assess the production of IFN-γ, lymphocytes were stimulated with the CEF peptides or mitogens in vitro in the presence of monesin. After 5 hr, cells were harvested and stained with other surface antigens. Cells were fixed and permeabilized in 250 μl BD cytofix/cytoperm solution for 20 min. at 4° C. Cells were then incubated in 50 μl of BD Perm/Wash solution containing PE-conjugated anti-IFN-γ antibody or appropriate negative control for 30 min at 4° C., washed twice, and fixed in paraformaldehyde.

### Cytotoxicity Assay

CD8⁺ T_{EM} and T_{CM} cells were used for antibody redirected cytotoxicity assay 14 days after anti-CD3 stimulation. Serial dilutions of T cells were incubated in duplicate with 1×10⁴ Fc receptor-expressing P815 target cells in the presence of 0.5□g/ml anti-CD3 and 100 u/ml IL-2. After 4 hours, plates were centrifuged and supernatant were collected. The levels of LDH were determined using a Roche cytotoxicity detection kit according to the manufacturer's instructions.

### Statistical Analysis

To assess the differences of phenotype, IFN-γ production and CD107a expression between BM and PB-derived CD8⁺ T cells, T_{EM} and T_{CM} subsets, QQ-plots were first performed, which showed that the normality assumptions are violated so that the paired t-test cannot be used. The Wilcoxon matched-pairs signed rank sum test (Altman, D. G., Practical statistics for medical research, Chapman and Hall/CRC, 1991) was used to calculate the p-values. The significance level was set at 5%. Calculations were implemented with S-PLUS (Copyright (c) 1988, 2003 Insightful Corp.; Academic Site Edition Version 6.2.1, Seattle, Wash., USA). The Student's t test was used to assess the statistical significance in the cytotoxicity assay.

### Results

### Demographic Information

Written informed consent was obtained from twenty patients with a diagnosis of OA who were scheduled to undergo total joint replacement. All participants were Caucasian with no known diagnosis of cancer (other than non-melanoma skin cancer), nor a history of an autoimmune disease. Participants ranged in age from 47 to 75 with an average age of 62.8. Twelve of these patients were male and 10 were female. Not all samples were used in each experiment.

The Phenotype of BM CD8 T Cells is Distinct from PB

The cellular composition of paired BM and PB lymphocytes from OA patients was first defined. CD3⁺ T cells comprised 36.55% of lymphocytes in BM, compared to 63.98% in PB. In comparison to paired PB samples, the proportion of NK cells was decreased in the BM lymphocyte population, while the percentage of CD19⁺ cells was increased. Specifically, 48.16% of CD3⁺ cells were CD8⁺ in BM, compared to only 26.03% in PB. Thus, the ratio of CD4⁺/CD8⁺ decreased from 2.94 in PB to 1.11 in BM (Table 2). These data indicate that the T cell fraction in BM is composed of a higher percentage of CD8⁺ cells than PB.

TABLE 2
Cellular composition of mononuclear cells in PB and BM

| | PB | BM |
|---|---|---|
| CD3⁺ | 63.98 ± 14.29 | 36.55 ± 19.05 ** |
| CD4⁺ | 70.54 ± 8.29 | 51.29 ± 7.76 ** |
| CD8⁺ | 26.03 ± 6.93 | 48.16 ± 8.95 ** |
| CD4⁺/CD8⁺ | 2.94 ± 0.99 | 1.11 ± 0.30 ** |
| CD3⁻CD56⁺ | 16.32 ± 8.90 | 4.67 ± 2.97 ** |
| CD19⁺ | 7.54 ± 2.13 | 18.86 ± 18.65 |

BM and PB mononuclear cells of OA patients (n = 9) were stained and analyzed for lymphocyte subset markers. The results are shown as percentage of CD3⁺, CD3⁻CD56⁺, CD19⁺ cells from lymphocyte, as well as percentage of CD4⁺ and CD8⁺ T cells from CD3⁺ T cells of PB and BM. Mean values ± SD are given.
** p < 0.005.

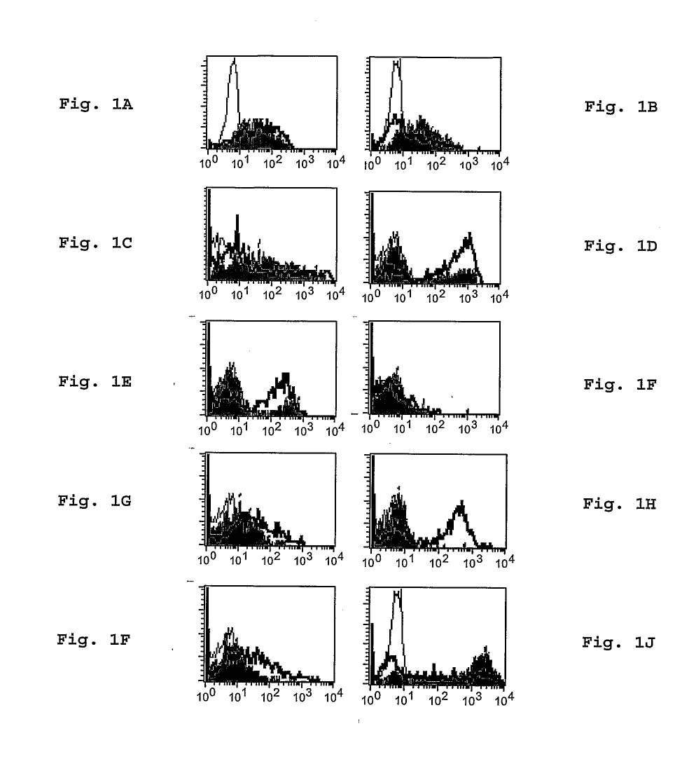

Next, the phenotypic differences between BM and PB CD8⁺ T cells. Flow cytometric analysis was performed to measure the expression of surface molecules, associated with CD8⁺ T cell activation and differentiation, in cells isolated from the PB and BM of patients with OA (Table 3). Despite similar expression of CD45RO and CD45RA, BM CD8⁺ T cells expressed higher levels of activation associated markers. In particular, the level of CD69 expression was profoundly up-regulated in CD8⁺ BM T cells (56.87±8.84) in comparison to those found in PB (10.97±9.82, P=0.0039). Small percentages of CD8⁺ T cells expressed CD25 in BM and PB. The frequency of CD8⁺ T cells that expressed CD62L was down regulated in BM in comparison to PB. The expression level of select costimulatory molecules was also examined. The frequency of CD27 expressing CD8⁺ T cells was higher in BM than in the PB (74.29±10.39 versus 53.36±24.15, P=0.0008). CD57, a marker associated with lymphocyte senescence was down regulated in CD8⁺ T cells of BM (Table 3 and FIG. 1J). These results reveal a unique phenotype of CD8⁺ T cells in BM, which demonstrate a profile (CD27^{Hi}CD62L^{Lo}CD69^{Hi}) that distinguishes them from naïve or resting T cells.

TABLE 3
Surface molecules on PB and BM CD8⁺ T cells

| | PB | BM | p-value | n |
|---|---|---|---|---|
| CD45RA | 60.03 ± 22.10 | 48.70 ± 17.83 | 0.0302 | 15 |
| CD45RO | 37.16 ± 15.47 | 43.63 ± 17.11 | 0.0984 | 17 |
| CD62L | 35.47 ± 17.81 | 21.64 ± 12.45 | 0.0005 | 17 |
| CD27 | 53.36 ± 24.15 | 74.29 ± 10.39 | 0.0008 | 17 |
| CD28 | 47.49 ± 23.21 | 58.48 ± 13.80 | 0.0730 | 15 |
| CD25 | 6.27 ± 5.79 | 4.53 ± 3.31 | 0.2500 | 9 |
| CD38 | 33.10 ± 12.91 | 49.67 ± 11.48 | 0.0008 | 17 |
| CD69 | 10.97 ± 9.82 | 56.87 ± 8.84 | 0.0039 | 9 |
| HLA-DR | 26.67 ± 18.34 | 51.30 ± 19.42 | 0.0003 | 17 |
| CD57 | 53.15 ± 19.33 | 33.33 ± 13.33 | 0.0003 | 17 |

PBMCs and BMCs were stained and analyzed for the expression of surface molecules known to correlate with cellular function. Results are shown as the percentage of positive cells gated on CD3⁺CD8⁺ cells. Mean values ± SD are given.

Viral Specific CD8⁺ T Cells in the BM have Potent Effector Function.

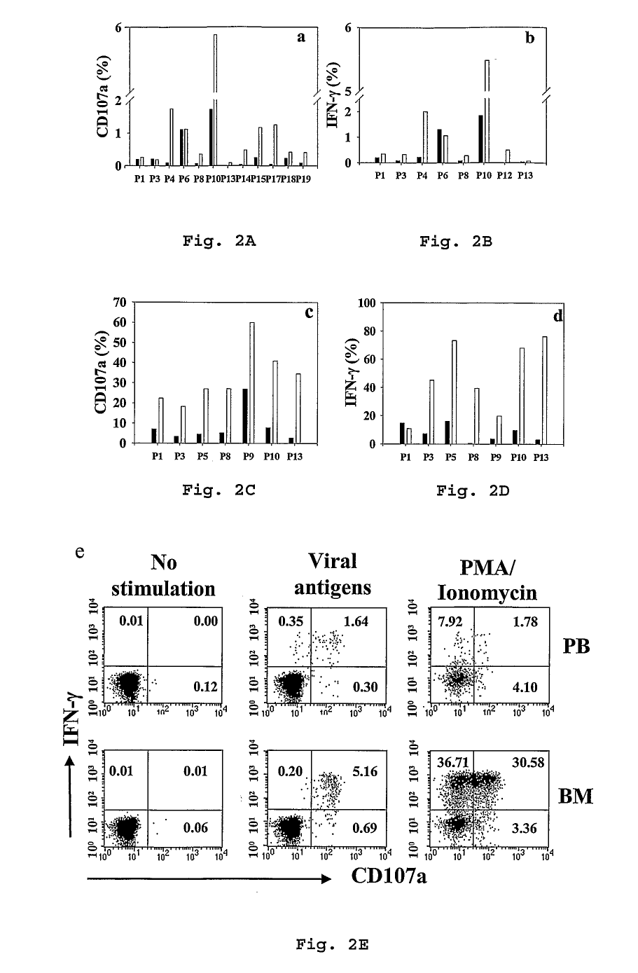

As CD8⁺ T cells in BM were found to express high levels of multiple activation markers, the present inventors sought to determine correlate functional status by evaluation of CD8⁺ T cell reactivity towards viral antigens. Specifically, a cocktail of CMV, EBV and Flu (CEF) peptides was used to stimulate BM and PB memory CD8⁺ T cells. After stimulation with CEF peptides in vitro, CD8⁺ memory T cells in both the BM and PB produced IFN-γ and expressed CD107a. However, BM memory CD8⁺ T cells were significantly more potent effectors. The frequency of IFN-γ producing memory CD8⁺ T cells among total CD8⁺ T cells from PB varied from approximately 0.03% to 1.84% versus 0.06% to 5.48% in BM (P=0.0547). The frequency of CD107a expressing cells varied from 0.00-1.72% in PB CD8⁺ T cells versus 0.09% to 5.94% in BM (P<0.0015). Compared to CD8⁺ T cells from the PB, the frequency of IFN-γ and CD107a producing cells in the BM, increased 2.7 fold and 3.3 fold, respectively (FIG. 2).

In order to evaluate the possibility that these findings were secondary to site specific differences in antigen presenting cell function in the PB and the BM, the reactivity of CD8⁺ T cells to TCR independent stimulation with phorbol ester PMA and calcium ionophore ionomycin was tested. CD8⁺ T cells in BM showed a more vigorous IFN-γ and CD107a response to PMA/iomomycin than correlate cells from paired PB. The frequency of PMA/ionomycin IFN-γ and CD107a expressing cells in BM varied between 10.92-73.27% and 18.21-60% respectively, while levels in PB were 0.65-16.01% and 2.71-26.93% respectively (FIG. 2). These data demonstrate that BM derived memory CD8⁺ T cells have an enhanced capacity to respond to viral recall antigens, and that this response is inherent in these cells, as these augmented functions are apparent even when cells are activated by strategies which bypass the TCR.

### Frequencies of Recall Antigen HLA Multimer-Binding CD8⁺ T Cells in BM

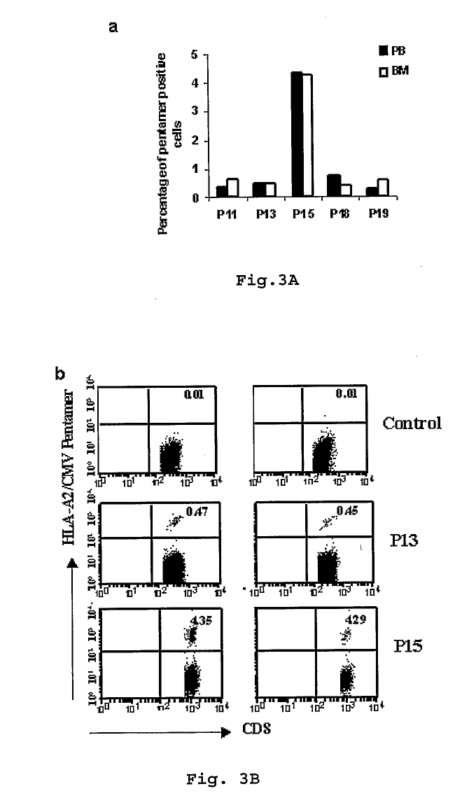

To assess whether the increased response of BM CD8⁺ T cells to CEF peptides was due to a higher percentage of antigen specific T cells, HLA-A2 CMV (NLVPMVATV; SEQ ID NO:7) pentameric complexes was used to quantify the proportion of CMV specific T cells. Five patients had pentamer staining CD8⁺ T cells (FIG. 3). The CD8⁺ T cells specific for CMV pp65 (495-503) represented 0.31%-4.35% of total CD8⁺ T cells in BM and 0.43-4.29% in the PB. These data suggest that the differential response of BM CD8⁺ cells to recall antigens is not due to an increased frequency of antigen specific precursor cells.

### BM CD8⁺ T Cells Rapidly Upregulate Perforin and Granzyme B in Response to TCR Stimulation.

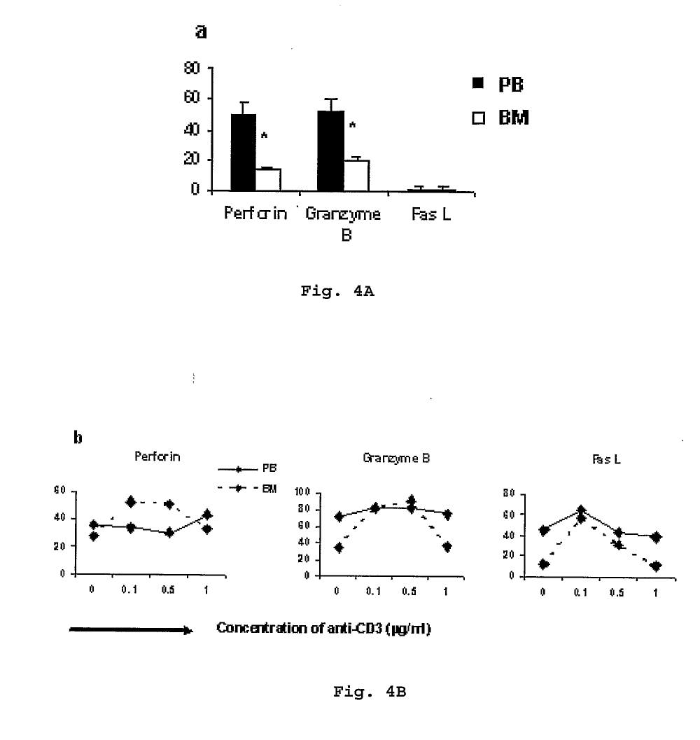

BM CD8⁺ T cells evidence enhanced production of IFNγ and CD107a in response to both antigen specific and TCR independent stimulation. In order to correlate our findings with other markers of granule release and cytotoxicity, we examined the expression of perforin, granzyme B and Fas L on CD8⁺ memory T cells from BM and PB. Interestingly, ex vivo expression of intracellular perforin and granzyme B was down-regulated in BM CD8⁺ T cells compared to correlate cells in PB (FIG. 4). Surface Fas L expression was negligible in both resting PB and BM CD8⁺ T cells. However, when cells were activated with the anti-CD3 mAb, to mimic TCR stimulation, BM derived memory CD8⁺ T cells evidenced profound upregulation of perforin, granzyme B and Fas L. These data demonstrate that BM CD8⁺ memory T cells have significantly less pre-stored perforin and granzyme B compared to memory CD8⁺ T cells in PB, yet rapidly upregulate these molecules in response to TCR stimulation.

### BM Contains an Increased Population of CD8⁺ T_{EM} Cells Versus PB.

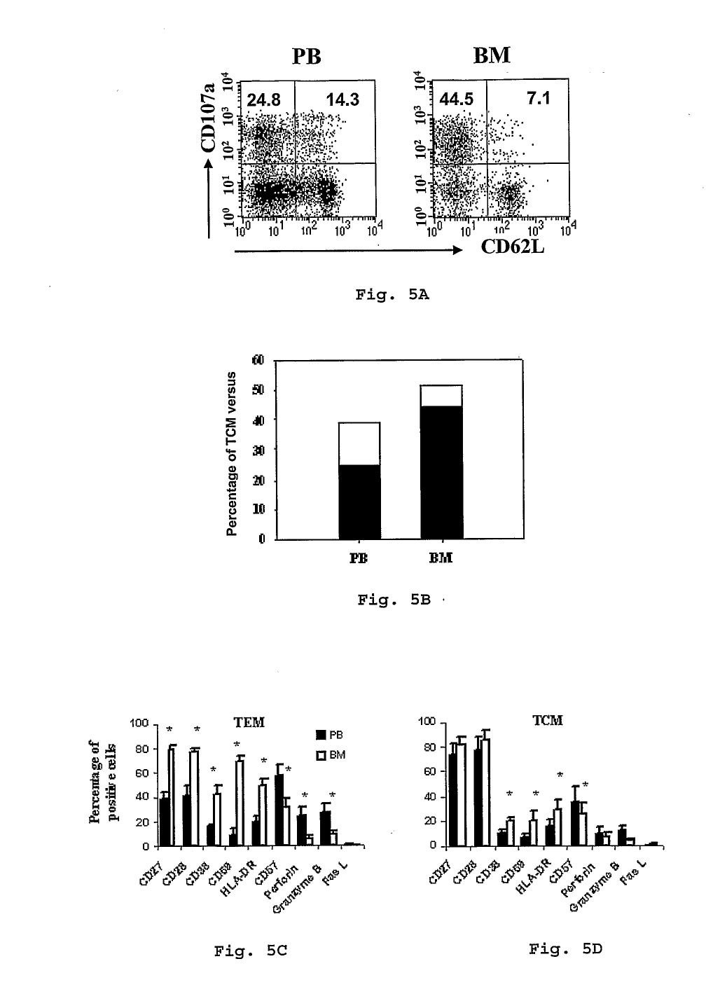

Memory T cells carrying distinct homing receptors participate in different types of immune responses and have different effector capacities. Specifically, these cells are categorized into T_{EM}, CD45RO⁺CD62L^{Lo}CCR7^{Lo}, and T_{CM}, CD45RO⁺CD62L^{Hi}CCR7^{Hi}, subsets based on both their homing characteristics and effector functions (Sallusto et al., 1999). The present inventors' analysis of the total complement of memory T cells in the BM and PB revealed significantly higher levels of CD38 and CD69 expression in the BM, with reduced levels of CD62L. The low expression of CD62L suggested that these activated cells might be part of the T_{EM} subset. To understand why CD8⁺ T cells in BM respond so vigorously to stimulation with common recall antigens, the T_{CM} and T_{EM} components of memory T cells in paired BM and PB samples were evaluated. While both CD8⁺ T_{CM} and T_{EM} subsets are present in the PB, the predominant subset represented in BM-derived memory CD8⁺ T cells is the T_{EM} (FIGS. 5A and B).

To assess the phenotypic characteristics of CD8⁺ T_{EM} cells, freshly isolated PBMCs and BMCs from 9 patients were stained with a panel of T cell differentiation and activation associated markers using four color flow cytometric analysis (FIGS. 5C and 5D). The CD8⁺ T_{EM} cell subset in BM, had increased expression of the CD27 and CD28 costimulatory molecules, in comparison to cells from PB (P<0.05). In contrast, the expression of these molecules on CD8⁺ T_{CM} cells was not significantly different between BM and PB. The activation markers including CD38, CD69 and HLA-DR were up-regulated in T_{EM} subset of BM in comparison to PB (all P values less than 0.05) while CD25 expression was not different between groups (data not shown). Similarly, CD8⁺ T_{CM} cells in the BM also expressed elevated levels of CD38, CD69 and HLA-DR (P<0.05). However, the level of expression of CD38, CD69 and HLA-DR in the T_{CM} subset was generally lower than that in the T_{EM} subset. Similar to the total CD8⁺ population, the expression level of both perforin and granzyme B were lower in T_{EM} subset of BM in comparison with correlate PB derived cells.

### The T_{EM} Component Contributes to the Augmented Antigen Specific CD8⁺ Recall Response to Viral Antigen in the BM.

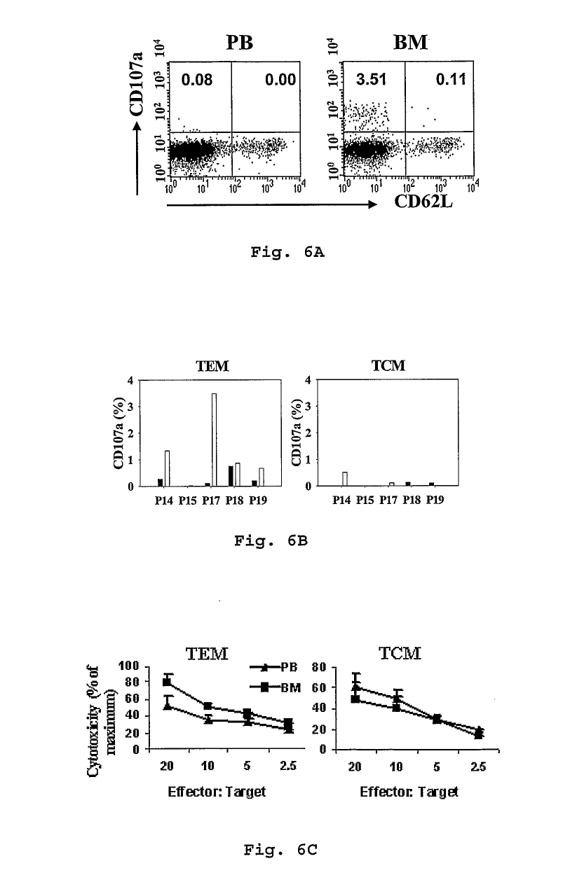

The increased CD8⁺ T_{EM} cell component in BM and its distinct phenotype and function prompted the present inventors to ask whether this particular subset of cells is responsible for the enhanced response of BM memory CD8⁺ T cells to recall antigens. To test this hypothesis, 5 paired BM and PB samples were used to evaluate granule exocytosis in T_{EM} and T_{CM} subsets in response to stimulation with viral recall antigens. BM and PB samples from 4 patients showed specific degranulation response to antigen. Following antigen stimulation, the majority of cells expressing CD107a, in both BM and PB, were T_{EM} cells (FIG. 6A). On a per cell basis, BM CD8⁺ T_{EM} cells demonstrated enhanced granule exocytosis in comparison to those found in the PB (FIG. 6B). These data demonstrate that not only is there an increased proportion of CD8⁺ T_{EM} cells in BM, but that on an individual basis, BM derived CD8⁺ T_{EM} cells have heightened antigen specific effector potential in comparison to their counterparts in the PB.

In order to directly evaluate T_{EM} killing, in vitro cytotoxicity of cultured T_{EM} cells from BM was compared with that of PB in an anti-CD3 redirected cytolysis assay. In 3 out of 4 experiments, BM derived T_{EM} cells demonstrated increased cytotoxicity in comparison to PB T_{EM} cells. Analogous studies employing T_{CM} cells from both BM and PB revealed no site specific differences (FIG. 6C). As expected, T cells failed to kill P815 targets when anti-CD3 mAb was not anchored on the target cells (Data not shown).

### Expression of Chemokine Receptors on Effector Memory CD8⁺ T Cells in BM

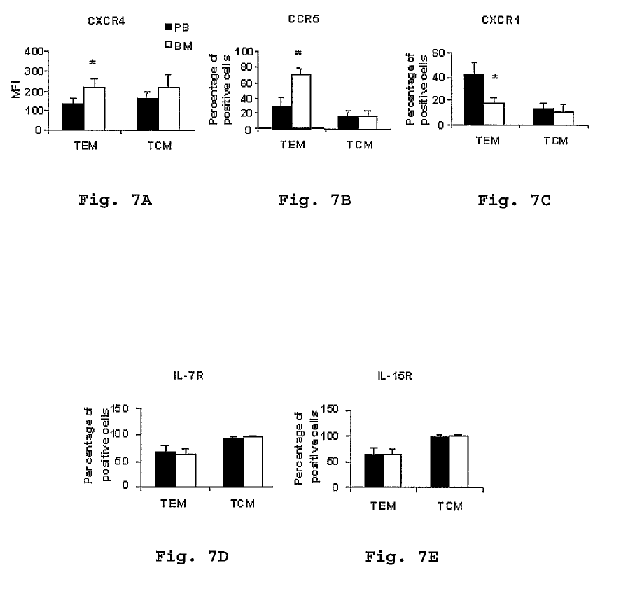

Finally, the present inventors queried why this unique population of T_{EM} cells is preferentially located in BM. Because chemokine receptors are involved in T cell homing and differentiation (Campbell et al., 2003), the expression of chemokine receptors on CD8⁺ T cells isolated from BM and PB was investigated and it was found that CCR5 and CXCR4 are upregulated in BM T_{EM}. Furthermore, CXCR1 is downregulated on T_{EM}, but not T_{CM} CD8⁺ T cells in BM compared to correlate cells in the PB. Interestingly, no difference in the expression of receptors for IL-7 or IL-15 that are critical for the proliferation and survival of CD8⁺ memory T cells was identified (FIG. 7) (Lee et al., 2005; Schluns et al., 2002; Maraskovsky et al., 1996; Ku et al., 2000; and Schluns et al., 2000). These data demonstrate that the patterns of chemokine receptor expression on BM T_{EM}, likely contribute to their accumulation within this compartment.

### Discussion

Previous studies in cancer patients have defined the human BM as a harbor for tumor antigen specific memory CD8⁺ T cells with potent effector function (Feuerer et al., 2001a, 2001b and 2003; Schmitz-Winnenthal et al., 2005; Choi et al., 2005; and Letsch et al., 2003). However, the phenotype and functional recall response of BM CD8⁺ memory T cells to viral antigens in humans remains poorly defined. In this study, a population of effector memory CD8⁺ T cells in the BM of patients with OA was characterized, which demonstrate a unique effector phenotype characterized by high levels of CD27, CD38, CD69, and HLA-DR, and enhanced recall responses to viral antigens. The description of the phenotypic signature and function of this enhanced memory CD8⁺ T cell population within the human BM provides a requisite link to pave the way for clinical translation.

The initial studies evaluated the overall population of memory CD8⁺ T cells within the BM of patients with OA. The findings are consistent with previous murine studies, demonstrating that BM derived CD8⁺ T cells have high expression of defined costimulatory and activation markers and preferentially respond to viral recall antigens (Slifka et al., 1997; and Di et al., 2002). This enhanced recall response does not appear to result from an increased percentage of antigen specific T cells in the BM, as pentamer staining with an HLA-A2 CMV restricted peptide, revealed similar percentages of antigen specific cells in both the BM and PB.

Importantly, the majority of memory T cells within the BM also express low levels of CD62L, a marker for effector memory T cells. Memory T cells can be divided into two subsets, T_{EM} and T_{CM}, based on their expression of CCR7 and CD62L. T_{EM} cells express high levels of activation molecules and effector molecules, low levels of costimulatory molecules, and exert immediate effector function. In contrast, T_{CM} cells express high levels of costimulatory molecules, low levels of effector molecules and possess high proliferative capacity (Sallusto et al., 2004)). Based on the relatively large population of T_{EM} within the human BM, the present inventors sought to determine if a unique T_{EM} population could account for the observed phenotypic and functional changes.

In comparison to correlate cells in the PB, BM derived T_{EM} express high levels of the CD27 and CD28 costimulatory molecules, which are postulated to serve as markers for antitumor and anti-viral protection, and whose loss defines end-stage T cell differentiation (Hamann et al., 1997; and Wherry et al., 2003). For example, tumor-specific T cells bearing a CD27⁺CD28⁺ phenotype, similar to the BM-derived CD8⁺ T cells described here, have recently been associated with successful adoptive immunotherapy in patients with metastatic melanoma (Dudley et al., 2002; and Powell et al., 2005). Similarly, in patients infected with HIV, CD27 positive CD8⁺ cells preferentially survive in vivo, proliferate following antigenic stimulation and are more resistant to apoptosis than CD27 negative cells (Ochensbein et al, 2004). The high levels of CD27/CD28 on BM CD8⁺ T_{EM}, suggest that this population of cells might be responsible for the potent effector function of BM memory T cells.

In addition to costimulatory molecules, BM derived CD8⁺ T_{EM} demonstrate upregulation of the CD38, CD69, and HLA-DR activation markers. Specifically, expression of these activation markers is enhanced in comparison to both PB T_{EM} and BM T_{CM}. The high expression of CD38, CD69 and HLA-DR, correlates well with previous studies which demonstrate that BM T cells are in a heightened state of activation (Mills et al., 1996; and Di et al., 2002) and are phenotypically distinct from memory cells in the PB (Feuerer et al., 2001b). These data suggest that BM derived memory T_{EM} cells are phenotypically unique, bearing a signature associated with enhanced functional activity.

In order to correlate the phenotypic signature of BM T_{EM} cells with effector function, CD107a expression was analyzed in a series of short term recall studies. These assays enabled the present inventors to monitor antigen specific degranulation response in defined CD8⁺ T cell subsets with minimal manipulation in vitro (Betts et al., 2003). In comparison to CD8⁺ T_{EM} cells in the PB, BM-derived CD8⁺ T_{EM} exhibit an increased expression of the degranulation marker CD107a in response to activating stimuli. Furthermore, despite lower resting levels of perforin and granzyme B, these BM-derived CD8⁺ effector memory T cells exhibit enhanced cytolytic capacity, exceeding that of the highly potent memory subset in PB.

In order to better understand why this unique population of CD8⁺ T_{EM} cells is preferentially located in the BM, specific patterns of chemokine and growth factor receptor expression were analyzed. These studies indicate that BM CD8⁺ T_{EM} have increased expression of CXCR4 and CCR5 and decreased expression of CXCR1. High expression of CXCR4 on BM CD8⁺ T_{EM} is likely physiologically relevant, as CXCL12, the ligand for CXCR4, is constitutively expressed by both BM stromal cells and the endothelium of BM microvessels (Bleul et al., 1996; and Peled et al., 1999a), and mediates the homing and localization of hematopoietic stem cells to the BM (Wright et al., 2002; and Peled et al., 1999b). Similarly, CCL3 and CCL5, both ligands for CCR5, are produced by BM fibroblasts (Brouty-Boyé et al., 1998). In contrast, CXCL8, the ligand for CXCR1, induces mobilization of hemotopoietic stem cells from BM. These data suggest that cognate interactions between CXCR4 and CCR5 and their ligands, stimulate T_{EM} migration to the BM, while the low levels of CXCR1 found on BM CD8⁺ T_{EM} might hinder exodus (Pruijt et al., 2002).

The preferential accumulation of CD8⁺ T_{EM} cells in the BM may also be due to the specific microenvironment. BM contains T cell survival factors, such as IL-7 and IL-15, which are recognized to induce antigen independent proliferation of memory T cells (Lee et al., 2005; Schluns et al., 2003; and Gutierrez-Ramos et al., 1992). Although, no difference in IL-7 or IL-15 receptor expression on CD8⁺ T_{EM} cells in BM compared to those in PB was found, recent evidence suggests that homeostatic proliferation to IL-15 might not depend on the expression of IL-15Rα (Burkett et al., 2003; and Schluns et al., 2004). Thus, these studies do not rule out the potential import of IL-7 or IL-15 on the homeostatic proliferation of BM T_{EM}.

Several limitations of this study are important to note. Specifically, OA is classically considered to result from mechanical degradation of the joint and synovial inflammation. However, new evidence suggests a potential role for autoimmune synovial attack (Benito et al., 2005; and Sakata et al., 2003). Based on the fact that specimens were harvested directly from the medullary canal and demonstrated a heightened response to non-synovial recall antigens, it is unlikely that the findings here are directly related to OA. However, the possibility that some of our observations are a result of the underlying disease process or are due to the fact that OA commonly occurs in the elderly cannot be completely eliminated. For example, a recent study has shown that the human BM contains an approximately equal proportion of both CD8⁺ T_{CM} and T_{EM} cells Mazo et al., 2005. The high percentage of T_{EM} versus T_{CM} in the BM of OA patients observed in this study maybe due to an age-associated change (Hong et al., 2004).

Taken together, the data in this study suggest that human BM is enriched with functionally enhanced population of CD8⁺ T_{EM} cells, which bear a hybrid phenotype (CD45RO^{Hi}, CD62L^{Lo}, CD27^{Hi}, CD69^{Hi}, CD38^{Hi}, Perforin^{Lo}), between classically defined T_{EM} and T_{CM} subsets. These BM T_{EM} are characteristic of cells at intermediate stages of differentiation, which may have the potential for self-renewal and homeostasis in the absence of antigen. While several elegant murine models have been described for studying T cell memory in BM (Masopust et al., 2001; Slifka et al., 1997; Jacob et al., 1999; and Weninger et al., 2001), to the best of the present inventors' knowledge, this is the first report to define the unique phenotype of BM CD8⁺ T_{EM} cells and characterize the viral recall response of CD8⁺ T_{EM} cells in human BM derived from patients with no known history of cancer or well-defined immunologically mediated disease. The phenotypic and functional data in this report provide a solid foundation for the use of BM-derived memory T cells in the treatment of infectious disease and cancer, and to promote their generation in vaccine design.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERENCES

Bai, L. et al. Cognate interactions between memory T cell and tumor antigen-presenting dendritic cells from bone marrow of breast cancer patients: Bidirectional cell stimulation, survival and antitumor activity in vivo. Int. J. Cancer 103, 73-83 (2003).
Beckhove, P. et al. —Specifically activated memory T cell subsets from cancer patients recognize and reject xenotransplanted autologous tumors. —Journal of Clinical Investigation July 2004; 114(1):67-76.
Benito et al., B. Synovial tissue inflammation in early and late osteoarthritis. Ann Rheum Dis (2005).
Betts, M. R. et al. Sensitive and viable identification of antigen-specific CD8+ T cells by a flow cytometric assay for degranulation. Journal of Immunological Methods 281, 65-78 (2003).
Bleul et al., A highly efficacious lymphocyte chemoattractant, stromal cell-derived factor 1 (SDF-1). J Exp Med.; 184:1101-1109 (1996).
Brouty-Boyé et al., Phenotypic diversity in human fibroblasts from myelometaplasic and non-myelometaplasic hematopoietic tissues. International Journal of Cancer.; 76:767-773 (1998).
Burkett et al., IL-15Ralpha expression on CD8+ T cells is dispensable for T cell memory. PNAS.; 100:4724-4729 (2003).
Campbell et al., Chemokines in the systemic organization of immunity. Immunological Reviews.; 195:58-71 (2003).
Chen, L. Co-inhibitory molecules of the B7-CD28 family in the control of T-cell immunity. Nature Reviews Immunology 4, 336-347 (2004).
Choi et al., Enrichment of functional CD8 memory T cells specific for MUC1 in bone marrow of patients with multiple myeloma. Blood.; 105:2132-2134 (2005).
Crossland et al., Greenberg P D, Cheever M A. T cells from tumor-immune mice nonspecifically expanded in vitro with anti-CD3 plus IL-2 retain specific function in vitro and can eradicate disseminated leukemia in vivo. J Immunol.; 146:4414-4420 (1991).
Currier, J. R. et al. A panel of MHC class I restricted viral peptides for use as a quality control for vaccine trial ELISPOT assays. Journal of Immunological Methods 260, 157 (2002).
Di Rosa, F. & Santoni, A. Bone marrow CD8 T cells are in a different activation state than those in the lymphoid periphery. Eur. J. Immunol. 32:1873-1880 (2002).
Dudley, M. E. et al. Cancer regression and autoimmunity in patients after clonal repopulation with antitumor lymphocytes. Science 298:850-854 (2002).
Feuerer, M. et al. Bone marrow as a priming site for T cell responses to blood-borne antigen. Native Medicine, 9:1151-1157 (2003).
Feuerer, M. et al. Enrichment of memory T cells and other profound immunological changes in the bone marrow from untreated breast cancer patients. International Journal of Cancer. 92:96-105 (2001).
Feuerer, M. et al. Therapy of human tumors in NOD/SCID mice with patient-derived reactivated memory T cells from bone marrow. Nature Medicine 7:452-458 (2001).
Gillison et al., Evidence for a casual association between human papillomavirus and a subset of head and neck cancers, Journal of the National Cancer Institute, 92:709-720 (2000)
Gillison et al., Human papillomavirus in head and neck squamous cell carcinoma: are some head and neck cancers a sexually transmitted disease? Current Opinion in Oncology, 11:191-1999 (1999)
Gutierrez-Ramos et al., Interleukin (IL1 to IL7) gene expression in fetal liver and bone marrow stromal clones: cytokine-mediated positive and negative regulation. Exp Hematol.; 20:986-990 (1992).
Hamann et al., Rep M H G, et al. Phenotypic and Functional Separation of Memory and Effector Human CD8+ T Cells. J Exp Med.; 186:1407-1418 (1997).
Hong et al., Age-associated changes in the frequency of naIve, memory and effector CD8+ T cells. Mechanisms of Ageing and Development.; 125:615 (2004).
Jacob, J. & Baltimore, D. Modeling T-cell memory by genetic marking of memory T cells in vivo. Nature 399, 593-7 (1999).
Kaech, S. M. & Ahmed, R. Memory CD8+ T cell differentiation: initial antigen encounter triggers a developmental program in naive cells. Nature Immunology 2, 415-422 (2001).
Kaech, S. M., Wherry, E. J. & Ahmed, R. Effector and memory T-cell differentiation: implications for vaccine development. Nature Reviews Immunology 2, 251-262 (2002).
Khazaie, K. et al. Persistence of dormant tumor cells in the bone marrow of tumor cell-vaccinated mice correlates with long-term immunological protection. Proceedings of the National Academy of Sciences of the United States of America. 91, 7430-4 (1994).
Klonowski, K. D. et al. Dynamics of Blood-Borne CD8 Memory T Cell Migration In Vivo. Immunity 20:551 (2004).
Ku et al., Control of Homeostasis of CD8+ Memory T Cells by Opposing Cytokines. Science.; 288:675-678 (2000).
Lee et al., Role of interleukin-7 in bone and T-cell homeostasis. Immunological Reviews.; 208:169 (2005).
Letsch et al., Bone Marrow Contains Melanoma-reactive CD8+ Effector T Cells and, Compared with Peripheral Blood, Enriched Numbers of Melanoma-reactive CD8+ Memory T Cells. Cancer Res.; 63:5582-5586 (2003).
Maraskovsky et al. Impaired survival and proliferation in IL-7 receptor-deficient peripheral T cells. J Immunol.; 157:5315-5323 (1996).
Masopust, D., Vezys, V., Marzo, A. L. & Lefrancois, L. Preferential Localization of Effector Memory Cells in Nonlymphoid Tissue. Science 291:2413-2417 (2001).
Mazo et al., Bone Marrow Is a Major Reservoir and Site of Recruitment for Central Memory CD8+ T Cells. Immunity.; 22:259 (2005).
Mills, K. C. et al. Immunologic phenotype and function in human bone marrow, blood stem cells and umbilical cord blood. Bone Marrow Transplantation 18:53-61 (1996).
Muller, M. et al. EblacZ tumor dormancy in bone marrow and lymph nodes: active control of proliferating tumor cells by CD8+ immune T cells. Cancer Res 58:5439-5446 (1998).
Ochensbein, A. et al. CD27 expression promotes long-term survival of functional effector-memory CD8+ cytotoxic T lymphocytes in HIV infected patients. J. Exp. Med. 200:1407-1417 (2004).
Peled et al., The chemokine SDF-1 stimulates integrin-mediated arrest of CD34+ cells on vascular endothelium under shear flow. J Clin Invest.; 104:1199-1211 (1999).
Peled et al. Dependence of Human Stem Cell Engraftment and Repopulation of NOD/SCID Mice on CXCR4. Science.; 283:845-848 (1999).
Powell, D. J., Dudley, M., Robbins, P. & Rosenberg, S. Transition of late-stage effector T cells to CD27+CD28+ tumor-reactive effector memory T cells in humans after adoptive transfer therapy. Blood 105:241-250 (2005).
Pruijt et al. Neutrophils are indispensable for hematopoietic stem cell mobilization induced by interleukin-8 in mice. PNAS.; 99:6228-6233 (2002).
Sakata, M. et al. Osteoarthritic articular chondrocytes stimulate autologous T cell responses in vitro. Clinical & Experimental Rheumatology 21:704-10 (2003).
Sallusto et al., Central memory and effector memory T cell subsets: Function, generation, and maintenance. Ann Rev Immunol.; 22:745-763 (2004).
Sallusto et al., Two subsets of memory T lymphocytes with distinct homing potentials and effector functions. Nature 402:708-712 (1999).
Schluns et al., Interleukin-7 mediates the homeostasis of na□ve and memory CD8 T cells in vivo. Nature Immunology; 1:426-432 (2000).
Schluns et al., Transregulation of memory CD8 T-cell proliferation by IL-15R{alpha}+ bone marrow-derived cells. Blood.; 103:988-994 (2004).
Schluns et al., Cytokine control of memory T-cell development and survival. Nature Reviews Immunology. 2003; 3:269-279.
Schluns et al., Cutting Edge: Requirement for IL-15 in the Generation of Primary and Memory Antigen-Specific CD8 T Cells. J Immunol.; 168:4827-4831 (2002).
Schmitz-Winnenthal et al. High Frequencies of Functional Tumor-Reactive T Cells in Bone Marrow and Blood of Pancreatic Cancer Patients. Cancer Res.; 65:10079-10087 (2005).
Slifka, M. K., Whitmire, J. K. & Ahmed, R. Bone Marrow Contains Virus-Specific Cytotoxic T Lymphocytes. Blood 90:2103-2108 (1997).
Strome et al., Squamous cell carcinoma of the tonsils: A molecular analysis of HPV associations, Clinical Cancer Research, 18:1093-1100 (2002)
van der Bruggen et al., A gene encoding an antigen recognized by cytolytic T lymphocytes on a human melanoma, Science, 254(5038):1643-7 (1991)
Veiga-Fernandes, H., Walter, U., Bourgeois, C., McLean, A. & Rocha, B. Response of naive and memory CD8+ T cells to antigen stimulation in vivo. Nature Immunology 1, 47-53 (2000).
Weninger et al., Migratory Properties of Naive, Effector, and Memory CD8+ T Cells. J Exp Med.; 194:953-966 (2001).
Weninger, W., Crowley, M. A., Manjunath, N. & von Andrian, U. H. Migratory Properties of Naive, Effector, and Memory CD8+ T Cells. J. Exp. Med. 194:953-966 (2001).
Wherry, E. J. et al. Lineage relationship and protective immunity of memory CD8 T cell subsets. Nature Immunology 4:225-34 (2003).
Wright D E, Bowman E P, Wagers A J, Butcher E C, Weissman I L. Hematopoietic Stem Cells Are Uniquely Selective in Their Migratory Response to Chemokines. J Exp Med.; 195:1145-1154 (2002).

1. A method for eliciting an effective immune response against a disease which generates a weak and insufficient immune response in a patient suffering from the disease, comprising administering an antigen associated with the weakly immunogenic disease or associated with the causative agent of the weakly immunogenic disease directly into the bone marrow of the patient to elicit an effective immune response against the weakly immunogenic disease.

2. The method of claim 1, wherein the weakly immunogenic disease is a cancer.

3. The method of claim 2, wherein the antigen is a tumor associated antigen or a peptide fragment thereof.

4. The method of claim 1, wherein the weakly immunogenic disease is a viral infection.

5. The method of claim 4, wherein the antigen is a viral antigen.

6. The method of claim 4, wherein the viral infection is influenza.

7. The method of claim 1, wherein the causative agent is an agent of bioterrorism.

8. The method of claim 7, wherein the weakly immunogenic disease is anthrax.

9. The method of claim 1, wherein the effective immune response against the weakly immunogenic disease.is elicited by activating effector memory T cells in the bone marrow which express elevated levels of CD27 and CD28 costimulatory molecules and CD38, CD69 and HLA-DR cell surface molecules and which express reduced levels of CD57.

10. A method for eliciting an effective immune response against a disease which generates a weak and insufficient immune response in a patient suffering from the disease, comprising administering an antigen associated with the weakly immunogenic disease or associated with the causative agent of the weakly immunogenic disease directly into the bone marrow of the patient to elicit an effective immune response against the weakly immunogenic disease by activating effector memory T cells in the bone marrow which express elevated levels of CD27 and CD28 costimulatory molecules and CD38, CD69 and HLA-DR cell surface molecules.

11. The method of claim 1a, wherein the activated effector memory T cells in the bone marrow express reduced levels of CDS7.

12. A method for priming T cells to become memory T cells in the bone marrow against a disease which generates a weak and insufficient immune response in a patient, comprising administering an antigen associated with the causative agent of the weakly immunogenic disease directly into the bone marrow of a patient in need thereof to primeoT cells in the bone marrow against the weakly immunogenic disease.

13. The method of claim 12, wherein the causative agent in an agent of bioterrorism to which the patient could later potentially be exposed.

14. The method claim 13, wherein the causative agent is Bacillus anthracis or its biotoxin.

15. The method of claim 13, wherein the causative agent is ebola virus or another hemorrhagic virus.

16. The method claim 12, wherein the causative agent is an influenza virus.

17. The method of claim 12, wherein the causative agent is a bird flu virus.

18. The method of claim 17, wherein the bird flu virus is strain HSN1.

19. The method of claim 12, wherein the memory T cells in the bone marrow primed against the weakly immunogenic disease are those which express elevated levels of CD27 and CD28 costimulatory molecules and CD38, CD69 and HLA-DR cell surface molecules and which express reduced levels of CD57.

20. A method for priming T cells to become memory T cells in the bone marrow against a disease which generates a weak and insufficient immune response in a patient, comprising administering an antigen associated with the causative agent of the weakly immunogenic disease directly into the bone marrow of a patient in need thereof to prime T cells in the bone marrow against the weakly immunogenic disease, wherein the memory T cells in the bone marrow primed against the weakly immunogenic disease are those which express elevated levels of CD27 and CD28 costimulatory molecules and CD38, CD69 and HLA-DR cell surface molecules.

21. The method of claim 20, wherein the primed memory T cells in the bone marrow express reduced levels of CD57.

22-23. (canceled)

24. An isolated population of human memory CDS+ T cells from the bone marrow which is in a hyperresponsive/heightened activation state and which demonstrates an effector phenotype characterized by elevated level of CD27 costimulatory molecule, elevated levels of CD3S, CD69 and HLA-DRT cell activation markers, and enhanced recall responses to viral antigens and tumor associated antigens.

25. The isolated population of claim 24, which is further characterized by elevated level of CD2S costimulatory molecule and reduced level of CD57.

26. The isolated population of claim 24, which is further characterized by enhanced cytolytic capacity.

27. The isolated population of claim 24, which is characterized by the phenotype CD45ROHi, CD62LLO, CD27Hi, CD69Hi, CD3 SHi, PerforinLo.


SEQUENCE LISTING (46 sequences)

SEQ ID NO: 1 - protein, 9 aa, Influenza A virus
VSDGGPNLY

SEQ ID NO: 2 - protein, 9 aa, Influenza A virus
CTELKLSDY

SEQ ID NO: 3 - protein, 9 aa, Influenza M virus
GILGFVFTL

SEQ ID NO: 4 - protein, 9 aa, Influenza A virus
FMYSDFHFI

SEQ ID NO: 5 - protein, 9 aa, Epstein Barr virus LMP2A
CLGGLLTMV

SEQ ID NO: 6 - protein, 9 aa, Epstein Barr virus BMLF1
GLCTLVAML

SEQ ID NO: 7 - protein, 9 aa, Cytomegalovirus pp65
NLVPMVATV

SEQ ID NO: 8 - protein, 9 aa, Influenza NP
KTGGPIYKR

SEQ ID NO: 9 - protein, 10 aa, Influenza NP
RVLSFIKGTK

SEQ ID NO: 10 - protein, 9 aa, Influenza A
ILRGSVAHK

SEQ ID NO: 11 - protein, 9 aa, Epstein Barr virus
RVRAYTYSK

SEQ ID NO: 12 - protein, 9 aa, Epstein Barr virus
RLRAEAQVK

SEQ ID NO: 13 - protein, 9 aa, Influenza M
SIIPSGPLK

SEQ ID NO: 14 - protein, 10 aa, Epstein Barr virus EBNA4NP
AVFDRKSDAK

SEQ ID NO: 15 - protein, 9 aa, Epstein Barr virus
IVTDFSVIK

SEQ ID NO: 16 - protein, 9 aa, Epstein Barr virus
ATIGTAMYK

SEQ ID NO: 17 - protein, 10 aa, Epstein Barr virus RTA
DYCNVLNKEF

SEQ ID NO: 18 - protein, 9 aa, Influenza NP
LPFDKTTVM

SEQ ID NO: 19 - protein, 9 aa, Epstein Barr virus
RPPIFIRRL

SEQ ID NO: 20 - protein, 9 aa, Influenza NP
ELRSRYWAI

SEQ ID NO: 21 - protein, 8 aa, Epstein Barr virus B2LF-1
RAKFKQLL

SEQ ID NO: 22 - protein, 9 aa, Epstein Barr virus EBNA3A
FLRGRAYGL

SEQ ID NO: 23 - protein, 9 aa, Epstein Barr virus EBNA3
QAKWRLQTL

SEQ ID NO: 24 - protein, 12 aa, Cytomegalovirus
SDEEEAIVAYTL

SEQ ID NO: 25 - protein, 9 aa, Influenza NP
SRYWAIRTR

SEQ ID NO: 26 - protein, 8 aa, Influenza M
ASCMGLIY

SEQ ID NO: 27 - protein, 9 aa, Epstein Barr virus EBNA3C
RRIYDLIEL

SEQ ID NO: 28 - protein, 9 aa, Epstein Barr virus EBNA3A
YPLHEQHGM

SEQ ID NO: 29 - protein, 9 aa, CMVpp65
IPSINVHHY

SEQ ID NO: 30 - protein, 10 aa, Epstein Barr virus
EENLLDFVRF

SEQ ID NO: 31 - protein, 10 aa, Cytomegalovirus
EFFWDANDIY

SEQ ID NO: 32 - protein, 10 aa, Cytomegalovirus
TPRVTGGGAM

SEQ ID NO: 33 - protein, 314 aa, Homo sapiens
  misc_feature: MAGE-A3
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 34 - protein, 9 aa, Homo sapiens
FLWGPRALV

SEQ ID NO: 35 - protein, 9 aa, Homo sapiens
  misc_feature: MAGE-A3
KVAELVHFL

SEQ ID NO: 36 - protein, 14 aa, Homo sapiens
AELVHFLLLKYRAR

SEQ ID NO: 37 - protein, 15 aa, Homo sapiens
TSYVKVLHHMVKISG

SEQ ID NO: 38 - protein, 12 aa, Homo sapiens
VIFSKASSSLQL

SEQ ID NO: 39 - protein, 98 aa, Human papillomavirus
MHGDTPTLHEYMLDLQPETTDLYCYEQLHDSSEEEDEIDGPAGQAEPDRAHYNIVTFCCKCDSTLRLCVQSTHVDIRTLEDLLMGTLGIVCPICSQKP

SEQ ID NO: 40 - protein, 9 aa, Human papillomavirus
MLDLQPETT

SEQ ID NO: 41 - protein, 8 aa, Human papillomavirus
TLGIVCPI

SEQ ID NO: 42 - protein, 22 aa, Human papillomavirus
PAGQAEPDRAHYNIVTFCCKCD

SEQ ID NO: 43 - protein, 9 aa, Human immunodeficiency virus
RKKRRQRRR

SEQ ID NO: 44 - protein, 4 aa, Artificial
  Chemically-synthesized furin cleavable linker
RVKR

SEQ ID NO: 45 - protein, 47 aa, Artificial
  Chemically-synthesized antigenic peptide
KVAELVHFLRVKRFLWGPRALVRVKRVIFSKASSSLQLRKKRRQRRR

SEQ ID NO: 46 - protein, 43 aa, Artificial
  Chemically-synthesized antigenic peptide
TLGIVXPIRVKRPAGQAEPDRAHYNIVTFXXKXDRKKRRQRRR